(19) United States
(12) Patent Application Publication
(10) Pub. No.: US 2011/0177104 A1
(43) Pub. Date: Jul. 21, 2011

(54) METHOD FOR SELECTIVE DEPLETION OF CD137 POSITIVE CELLS USING ANTI-CD137 ANTIBODY-TOXIN COMPLEX

(76) Inventors: Byung Suk Kwon, Ulsan (KR); Hong Rae Cho, Ulsan (KR); Sang Chul Lee, Ulsan (KR); Seon Gyeong Lee, Ulsan (KR); Eun Hwa Kim, Ulsan (KR); Hye Jeong Kim, Ulsan (KR)

(21) Appl. No.: 12/895,415

(22) Filed: Sep. 30, 2010

(30) Foreign Application Priority Data
Jan. 19, 2010 (KR) 10-2010-0004604

(51) Int. Cl. A61K 39/395; A61P 35/00; A61P 37/06; A61P 29/00; C12N 5/0781; C12N 5/0783; C12N 5/0784; C12N 5/0786; C12N 5/09; C12N 5/0787

(52) U.S. Cl. 424/183.1; 424/178.1; 435/375

(57) ABSTRACT

The present invention relates to method for depletion of CD137 positive cells using an anti-CD137 antibody-toxin complex, and more particularly, to a method for selective depletion of CD137 positive cells, comprising the step of contacting an anti-CD137 antibody-toxin complex with the CD137 positive cells. The method for selective depletion of CD137 positive cells in accordance with the present invention can be useful to prevent or treat various diseases including immune diseases mediated by the activation of the CD137 positive cells because this method is excellent in delivering a complex of an anti-CD137 antibody, specific to CD137 molecules, and a toxin to CD137 expressing cells and selectively killing the CD137 positive cells alone and is also excellent in suppressing cell proliferation.

### FIELD OF THE INVENTION

The present invention relates to a method for selective depletion of CD137 positive cells using an anti-CD137 antibody-toxin complex, and more particularly, to a method that selectively delivers a complex of an anti-CD137 antibody and a toxin to CD137 positive cells expressing CD137 and effectively depletes the CD137 positive cells by the toxin delivered into the cells.

### BACKGROUND OF THE INVENTION

In general, an immune response is induced by various processes. In particular, the process of immune response to T cells in vivo will be described as follows. First, an antigen present outside the cells is internalized by antigen-presenting cells and degraded, and the remainder forms a complex with a class II molecule of the major histocompatibility complex (MHC) formed within the cells. The resulting complex, after migrating to the outer surface of the antigen presenting cell, is exposed to the outside and recognized by a helper T cell antigen receptor, triggering an antigen-specific immune response. On the other hand, when an antigen, e.g., a viral antigen, is produced within a cell, it is partially degraded in the cell and the remainder forms a complex with a MHC class I molecule. The resulting complex moves to the outer surface of the antigen-presenting cell and an antigen-specific cellular immune response is initiated by the recognition of the complex by an antigen receptor of a cytotoxic T cell. Subsequently, the T and antigen-presenting cells enter the initial stage of activation where new molecules are expressed on the surfaces of the cells. The expressed molecules bind to each other and this binding accelerates the activation of the T and antigen-presenting cells, thereby promoting various immune responses.

In such an immune response process, the new molecules expressed on the surfaces of the T and antigen-presenting cells are called accessory molecules. Representative accessory molecules include B7-1, B7-2, CD28, CTLA4, CD40, CD40 ligand, and CD 137 (Goodwin et al., Eur. J. Immunol., 23, 2631 (1993)).

CD137, one of the accessory molecules mentioned above, was originally found as a protein expressed by activated rat T cells (Kwon, et al., Proc. Natl. Acad. Sci. U.S.A., 84, 2896-2900 (1987); and Kwon and Weissman, Proc. Natl. Acad. Sci. U.S.A. 86, 1963-1967 (1989)) and subsequently demonstrated to encode a member of the tumor necrosis factor (TNF) receptor family of total membrane proteins (Mallett and Barclay, A. N., Immunol. Today, 12, 220-222 (1991)). This receptor family is characterized by the presence of cysteine-rich motifs in the extracellular domain. Other members of this family include NGFR, CD40, OX-40, CD27, TNFR-I, TNFR-II, Fas and CD30 (Smith, et al., Cell, 76, 959-962 (1994); and Beutler, B. and VanHuffel, C, Science. 264, 667-668 (1994)).

CD137 is a 55 kDa homodimer and is expressed on a variety of rat T cell lines, thymocytes and mature T cells upon activation with concanavalin A (Con A), phytohemagglutinin (PHA) and ionomycin, or anti-CD3i (Kwon, et al., Proc. Natl. Acad. Sci. U.S.A. 86, 1963-1967 (1989); Pollok, et al., J. Immunol. 150. 771-781 (1993)). A part of CD137 is present inside the cells and binds to p56lck, one of protein kinases, and this suggests that CD137 plays an important role in intracellular signaling (Kim, et al., J. Immunol., 151, 1255-1262 (1993)). Recently, it was found that CD137 molecules are stimulatory molecules induced by the activation of T cells and that the expression of CD137 is antigen-specific and selective (Greenberg, Blood. 2007 Jul., 1, 110 (1), 201-210; Greenberg, Cytometry. A. 2008, 73a (11), 1043-1049).

One of the most important functions of the immune system is to recognize self-antigens and discriminate them from foreign-antigens. Under normal conditions, the immune system responds not to self antigens but only to foreign antigens. However, breakdown of such normal immunological tolerance may lead to a pathological condition wherein the immune system recognizes self-antigens as foreign-antigens, thereby destroying native cells, tissues and organs. Such diseases are collectively called autoimmune diseases.

The pathogenesis of autoimmune diseases has not been found yet but there have been only a few fragmentary studies on the high incidence rate of a specific autoimmune disease in a race carrying a specific leukocyte genotype and on whether a specific type of self-antigen is associated with a specific autoimmune disease, and the ultimate cause of breakdown of immunological tolerance and a method of suppressing this breakdown have not be clearly identified.

Accordingly, currently available methods to treat autoimmune diseases involves, rather than fundamental treatment of autoimmune diseases, administration of an anti-inflammatory agent to suppress inflammation caused by the autoimmune response, direct administration of methotrexate which is cytotoxic to actively proliferating cells, radiotherapy or thoracic duct drainage to suppress excessive immune responses, and clinical use of immunosuppressive anti-lymphocyte serum (ASL) such as anti-lymphocyte globulin (ALG) and anti-thymocyte globulin (ATG).

These treatments of autoimmune diseases may show some short-term effectiveness, but, as mentioned above, immunosuppressive therapies may affect normal immune cells and eventually damage the normal cells. Therefore, there is a disadvantage that an immunosuppressive agent cannot act selectively on activated immune cells directly associated with immune responses.

As such, new methods of treatment of autoimmune diseases are being developed, such as methods of using an immunosuppressive agent or an antibody capable of depleting immune cells. The use of antibodies has the advantage of targeting only specific target cells without nonspecific cell damage. Conventional techniques using antibodies for the treatment of autoimmune disease include Korean Patent Publication No. 2009-0059149, which discloses a humanized anti-human CD19 antibody, the oncology of the antibody, and its use in the transplantation and in the treatment of autoimmune diseases, Korean Patent Publication No. 2007-0019727, which discloses a method of preventing autoimmune diseases using a CD20 antibody, and Korean Patent Publication No. 2009-0122910 discloses a pharmaceutical composition comprising an anti-CD6 monoclonal antibody used in the diagnosis and treatment of rheumatoid arthritis.

Nonetheless, the conventional antibodies used for autoimmune disease therapy has faced some problems associated with side effects, such as second infection, resistance, and toxicity on normal cells, because of their strong immunosuppressive effects.

Therefore, there is an urgent need to develop a new method of therapy that can treats autoimmune diseases by effectively suppressing immune responses to specific antigens or depleting activated immune cells directly associated with immune responses without causing these side effects.

### SUMMARY OF THE INVENTION

It is, therefore, an object of the present invention to provide a method for depletion of CD137 positive cells in vitro and in vivo, including the step of contacting an anti-CD137 antibody-toxin complex with CD137 positive cells, which can effectively prevent and treat diseases caused by the activation of the CD137 expressing cells.

To accomplish the aforementioned object of the present invention, the present invention provides a method for depletion of CD137 positive cells in vitro and in vivo, including the step of contacting an anti-CD137 antibody-toxin complex with the CD137 positive cells expressing CD137.

In accordance with one embodiment of the present invention, the anti-CD137 antibody may be an agonist antibody or antagonist antibody against CD137 molecules.

In accordance with one embodiment of the present invention, the toxin may be a chemotherapeutic agent selected from the group consisting of cyclophosphamide, melphalan, mitomycin C, bizelesin, cisplatin, doxorubicin, etoposide, mitoxantrone, SN-38, Et-743, actinomycin D, bleomycin, TLK286, SGN-15 and fludarabin; a Type I ribosome-inactivating protein selected from the group consisting of agrostin, b-32, bouganin, camphorin, curcin, gelonin, JIP60, momordin, PAP (pokeweed antiviral protein), saporin and trichosanthin; a Type II ribosome-inactivating protein selected from the group consisting of abrin, ricin, mistletoe lectin I, modeccin, volkensin, RIP, lanceolin, stenodactylin, aralin and riproximin; diphtheria toxin; or venom toxin.

In accordance with one embodiment of the present invention, the anti-CD137 antibody-toxin complex may promote apoptosis of the CD137 positive cells or suppress proliferation of the CD137 positive cells.

In accordance with one embodiment of the present invention, the CD137 positive cells may be associated with a disease selected from the group consisting of autoimmune diseases, graft versus host diseases, transplantation, cancer, and inflammatory diseases.

In accordance with one embodiment of the present invention, the CD137 positive cells are activated cells expressing CD137, and may be selected from the group consisting of T cells, B-cells, dendritic cells, natural killer (NK) cells, macrophages, cancer cells, and myeloid cells containing neutrophils, basophils, and eosinophils.

In accordance with one embodiment of the present invention, the anti-CD137 antibody-toxin complex may enter the cells by endocytosis when contacted with the CD137 positive cells.

In accordance with one embodiment of the present invention, the toxin binds to the anti-CD137 antibody (primary antibody) or to a secondary antibody to the anti-CD137 antibody.

In accordance with one embodiment of the present invention, the CD137 positive cells may be treated with the anti-CD137 antibody-toxin complex at a concentration of 0.1 to 5.0 μg/ml.

The method for selective depletion of CD137 positive cells in accordance with the present invention can be useful to prevent or treat various diseases including immune diseases mediated by the activation of the CD137 positive cells because this method is excellent in delivering a complex of an anti-CD137 antibody, specific to CD137 molecules, and a toxin to CD137 expressing cells and selectively killing the CD137 positive cells alone and is also excellent in suppressing cell proliferation.

### BRIEF DESCRIPTION OF THE DRAWINGS

The above and other objects and features of the present invention will become apparent from the following description of preferred embodiments given in conjunction with the accompanying drawings, in which:

FIG. 1 is a fluorescence microscopic picture showing changes in the intracellular location of an anti-CD137 antibody over time after CD137 expressing cells are treated with the PE-conjugated anti-CD137 antibody;

FIG. 2a shows the expression of CD137 in T cells after peripheral blood mononuclear cells isolated from a human and a primate are cultured with an anti-CD3 antibody for 24 hours and reacted with a PE-conjugated anti-CD137 antibody for 30 minutes;

FIG. 2b shows the binding of an anti-CD137 antibody and CD137 in human peripheral blood mononuclear cells and the intracellular location of the anti-CD137 antibody over time;

FIG. 2c shows the binding of an anti-CD137 antibody and CD137 in primate peripheral blood mononuclear cells and the intracellular location of the anti-CD137antibody over time;

FIG. 3a is a graph showing the isolation and purification of an anti-CD137 antibody-doxorubicin complex, prepared in one embodiment of the present invention, using FPLC;

FIG. 3b shows the level of binding to CD137 molecules by staining CD137 expressing T cells with FITC-labeled anti-CD137 antibody-doxorubicin conjugates;

FIG. 4a shows a schematic diagram for measuring the effect of cell apoptosis in vitro by the anti-CD137 antibody-doxorubicin complex prepared in one embodiment of the present invention;

FIG. 4b shows the results obtained by measuring the effect of apoptosis of CD137 positive cells by the anti-CD137 antibody-doxorubicin complex.

FIG. 5a is a graph showing the results obtained, through Annexin V staining, by comparing the levels of cell apoptosis by the anti-CD137 antibody-toxin complex in accordance with the present invention between CD137 expressing cells and genetically CD137-deficient cells;

FIG. 5b is a graph comparing the levels of cell proliferation after CD137 expressing T cells are treated with an anti-CD137 antibody, an anti-C137 antibody-doxorubicin complex, and doxorubicin, respectively;

FIG. 5c shows the level of cell apoptosis, through Annexin V staining, after CD137 positive cells are treated with the FITC-labeled anti-CD137 antibody-toxin complex of the present invention.

FIG. 6 shows the results obtained by measuring the levels of CD137 expression in spleen and lymph node T cells of an acute GVHD-induced animal model by a flow cytometry;

FIG. 7a is a graph showing changes in body weight over time after the anti-CD137 antibody-doxorubicin complex prepared in one embodiment of the present invention was intraperitoneally injected to acute GVHD-induced mice;

FIG. 7b is a graph showing the survival rate of the mice;

FIG. 8 is a graph comparing the levels of cell apoptosis measured by a flow cytometry after EL-4 cells transfected with CD137 are treated with rat IgG, anti-CD137 antibody, rat IgG+anti-rat IgG-saporin complex, and anti-CD137 antibody+anti-rat IgG-saporin complex;

FIG. 9 is a graph showing the levels of cell apoptosis measured after immune cells isolated from mouse spleen are treated with an anti-CD3 antibody to activate the immune cells, the cells are treated with rat IgG, anti-CD137 antibody, rat IgG+anti-rat IgG-saporin complex, and anti-CD137 antibody+anti-rat IgG-saporin complex, and the cells are collected and stained with PE-Cys-anti-CD4 antibody, PE-anti-CD8 antibody, and FITC-Annexin V;

FIG. 10 is a graph showing the levels of cell apoptosis measured by a flow cytometry after monocytes isolated from human peripheral blood are treated with an anti-CD3 antibody, treated with anti-CD137 antibodies (agonist antibody and antagonist antibody), anti-CD137 antibody (agonistic antibody)+anti-mouse IgG-saporin complex, and anti-CD137 antibody (antagonistic antibody)+anti-mouse IgG-saporin complex, and then cultured;

FIG. 11 is a graph showing the levels of cell apoptosis measured by a flow cytometry after irradiated APCs isolated from BDF1 mice and mouse T cells are mixed and cultured together with an anti-CD3 antibody in cell culture fluid, and then the cultured cells are treated and cultured with an anti-rat IgG-saporin complex or an anti-goat IgG-saporin complex; and

FIG. 12 is a graph showing the levels of cell apoptosis measured by a flow cytometry after irradiated APCs isolated from human monocytes from different donors and T cells are mixed and cultured together with an anti-CD3 antibody in cell culture fluid, and then the cultured cells are treated and cultured with an anti-rat IgG-saporin complex or an anti-goat IgG-saporin complex.

### DETAILED DESCRIPTION OF THE EMBODIMENTS

The present inventors used CD137 molecules expressed in CD137 positive cells in order to develop a method for the treatment or prevention of diseases caused by the activation of CD137 expressing cells because the CD137 molecules are characterized in that the expression of the CD137 molecules is antigen-specific and selective.

As such, the present inventors have developed a method that delivers a complex, formed by binding a toxin to an antibody to CD137 molecules antigen-specifically expressed on CD137 positive cells, to target cells (i.e., CD137 expressing cells) and effectively depletes (induces apoptosis or inactivation) the target cells by the delivered toxin.

Accordingly, the present invention is characterized in that it provides a method for depletion of CD137 positive cells in vitro and in vivo, including the step of contacting an anti-CD137 antibody-toxin complex with the CD137 positive cells.

In the present invention, the anti-CD137 antibody may be a polypeptide capable of selectively recognizing and binding to CD137 molecules, or an agonist antibody or antagonist antibody to the CD137 molecules.

As used herein, the term “agonist antibody” refers to an antibody playing the role of promoting or activating an action induced by an antigen-antibody reaction, which binds to a specific molecule on the surface of a cell or inside the cell to induce a biological action in the cell by intracellular signaling. For example, if an agonist antibody binds to a CD137 molecule, one or more biological functions caused by the CD137 molecule in the cell can be improved.

The term “antagonist antibody” refers to an antibody which binds to a specific molecule on the surface of a cell or inside the cell to suppress biological functions or activation in the cell generated by the binding between an agonist antibody and a ligand. In the present invention, the antagonist antibody binds to CD137 molecules present on the surface of a cell to reduce or suppress one more biological functions caused by the CD137 molecules in the cell.

An antibody to the CD137 available in the present invention can be used irrespective of whether the antibody is agonistic or antagonistic because the method for depletion of CD137 positive cells is characterized in that an antibody binding to CD137 molecules is used to bind a toxin to the antibody, the complex is delivered into the cells, and the cells are depleted by inducing the inactivation or apoptosis of the CD137 positive cells by the toxin. Accordingly, the anti-CD137 antibody available in the present invention may be an agonist antibody or antagonist antibody to the CD137 molecules, preferably, an antagonist antibody capable of reducing or suppressing one or more biological functions caused by the CD137 molecules in the cells.

Moreover, CD137 of the present invention comprises CD137 of various mammals including human beings, but not limited thereto. Any anti-CD137 antibody to the CD137 used in the present invention can be used if it is commercially available, and it may be produced or isolated from mammals other than humans. In one embodiment of the present invention, an anti-CD137 monoclonal antibody provided from Dr. Mittler of Emory University was used.

In the present invention, a complex of an anti-CD137 antibody and a toxin was prepared as a substance for preventing or treating diseases mediated by activated CD137 positive cells. The toxin is a substance capable of suppressing or reducing the activation of the cells or capable of inducing apoptosis the cells, including a chemical treating agent, an enzyme inhibitor, a radionuclide, a bacterial toxin, etc. Preferably, the toxin may be a chemotherapeutic agent selected from the group consisting of cyclophosphamide, melphalan, mitomycin C, bizelesin, cisplatin, doxorubicin, etoposide, mitoxantrone, SN-38, Et-743, actinomycin D, bleomycin, TLK286, SGN-15 and fludarabin; a Type I ribosome-inactivating protein selected from the group consisting of agrostin, b-32, bouganin, camphorin, curcin, gelonin, JIP60, momordin, PAP (pokeweed antiviral protein), saporin and trichosanthin; a Type II ribosome-inactivating protein selected from the group consisting of abrin, ricin, mistletoe lectin I, modeccin, volkensin, RIP, lanceolin, stenodactylin, aralin and riproximin; diphtheria toxin or venom toxin. And more preferably, the toxin may be doxorubicin or saporin.

In particular, the doxorubicin used in one embodiment of the present invention is a substance capable of killing a cell by damaging DNA, which is used as an antitumor agent for lung cancer, digestive system cancer, bladder cancer, etc., and the saporin is a ribosome inactivating protein that inactivates ribosome when it enters the cytoplasm and thus kills the cells by stopping protein biosynthesis.

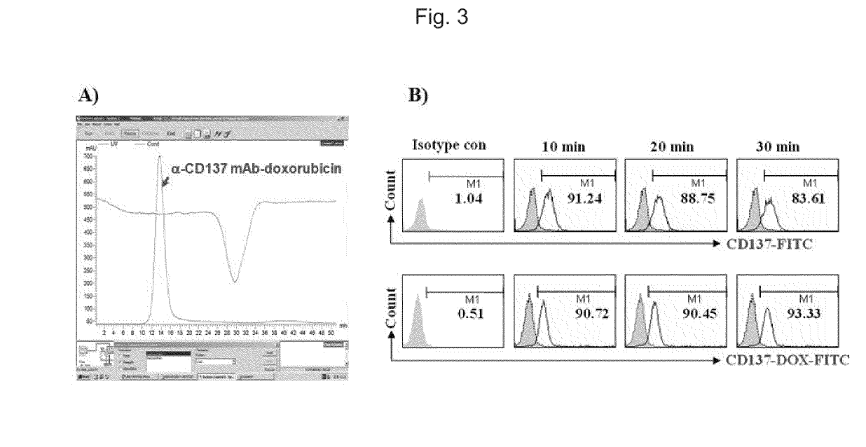

In one embodiment of the present invention, a complex of an anti-CD137 antibody-doxorubicin or saporin as a toxin was prepared, and FIG. 3a shows a result of the isolation and purification of the anti-CD137 antibody-doxorubicin complex by an FPLC method. Moreover, in order to the prepared complex to enter the cell by antigen-antibody binding, the complex has to bind to a CD137 molecule. As a result of analysis of the binding strength of the complex to the CD137 molecule, it was found that, when doxorubicin was conjugated to the anti-CD137 antibody, the complex was normally bound to the CD137 molecule (see FIG. 3b).

In the present invention, the anti-CD137 antibody-toxin complex can be prepared by using a well-known method of binding a chemical compound to an antibody, and the toxin may bind to a primary antibody to CD137 or a secondary antibody to the primary antibody.

In one embodiment of the present invention, a complex of an anti-CD137-monoclonal antibody, i.e., primary antibody, and doxorubicin was prepared, and a complex of a secondary antibody to the anti-CD137-monoclonal antibody and saporin was prepared.

Moreover, the present inventors investigated if the anti-CD137 antibody-toxin complex prepared by the above method of the present invention could be effectively delivered to a target cell before the determination of whether the complex could suppress the activation of CD137 positive cells or not.

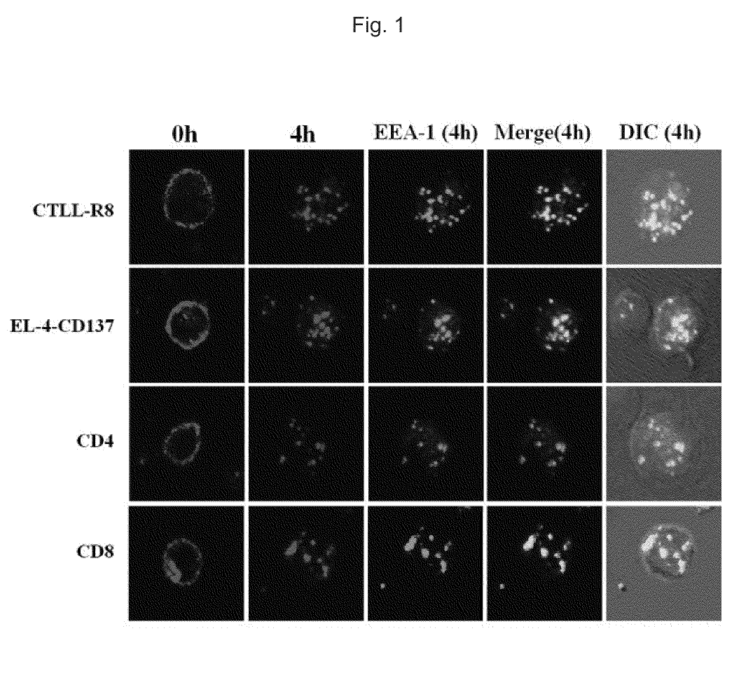

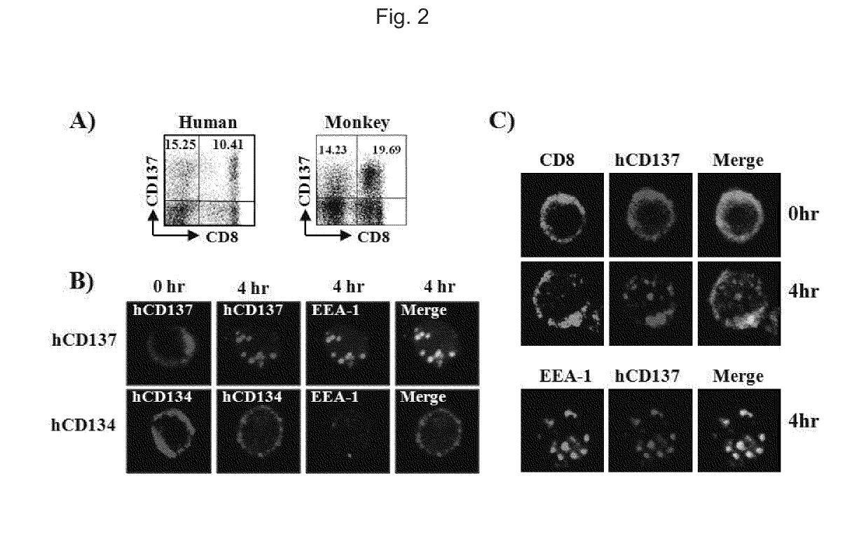

That is, in accordance with one embodiment of the present invention, in order to use the anti-CD137 antibody for the selective depletion of the CD137 positive cells, the anti-CD137 antibody has to specifically bind to CD137 and then be internalized into the cells. To confirm this, a fluorescence-labeled anti-CD137 antibody was cultured with CD137 expressed murine cell lines, and the intracellular location of the anti-CD137 antibody was observed over time. As a result, it was found that the anti-CD137 antibody present on the cell surface at incubation time 0 was internalized into the cells over time and its internalization into the cells was achieved by using an endocytosis marker EEA-1 (see FIG. 1). Also, the same result was observed in human and primate T cells, as well as the mouse T cells, for the internalization of the anti-CD137 antibody into the cells after binding to CD137 (see FIGS. 2a to 2c).

Accordingly, the present inventors found out that, in the case of the anti-CD137 antibody used in the present invention, if the anti-CD137 antibody binds to CD137 in CD137 expressing cells, it is internalized into the cells by endocytosis, and that the prepared anti-CD137 antibody-toxin complex, also, is easily internalized into the cells by endocytosis.

Therefore, the present invention provides a method of delivering the toxin to the CD137 positive cells expressing CD137 by using the anti-CD137 antibody-toxin complex.

Moreover, the anti-CD137 antibody-toxin complex prepared in the present invention is characterized in that it promotes the apoptosis of the CD137 positive cells expressing CD137 or suppresses the proliferation of the CD137 positive cells.

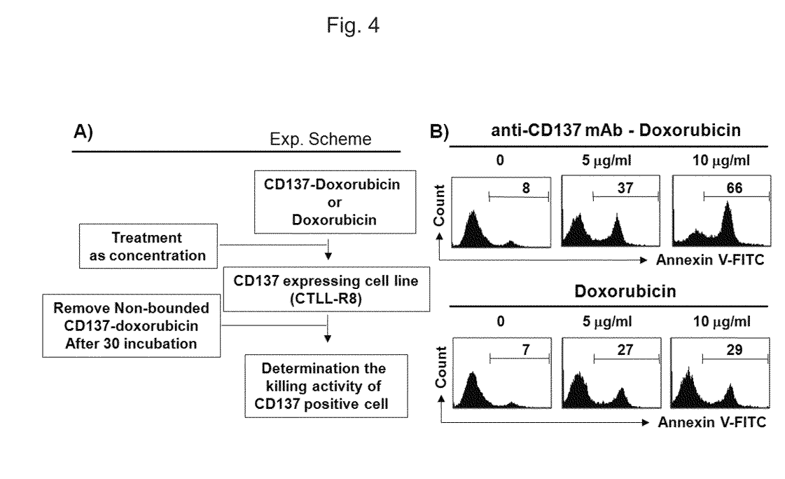

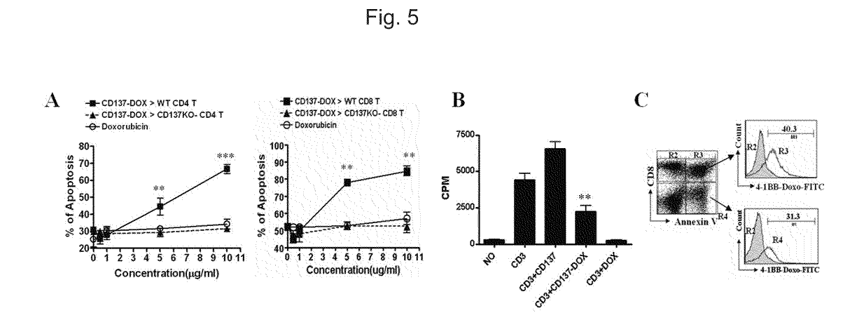

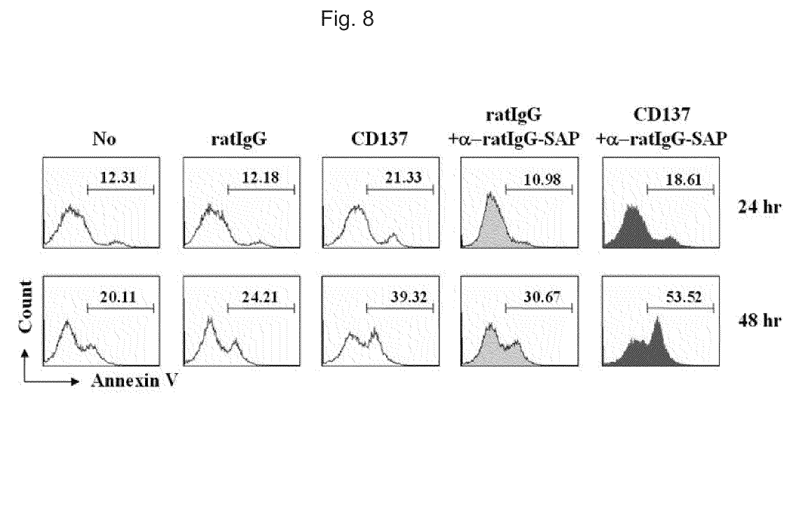

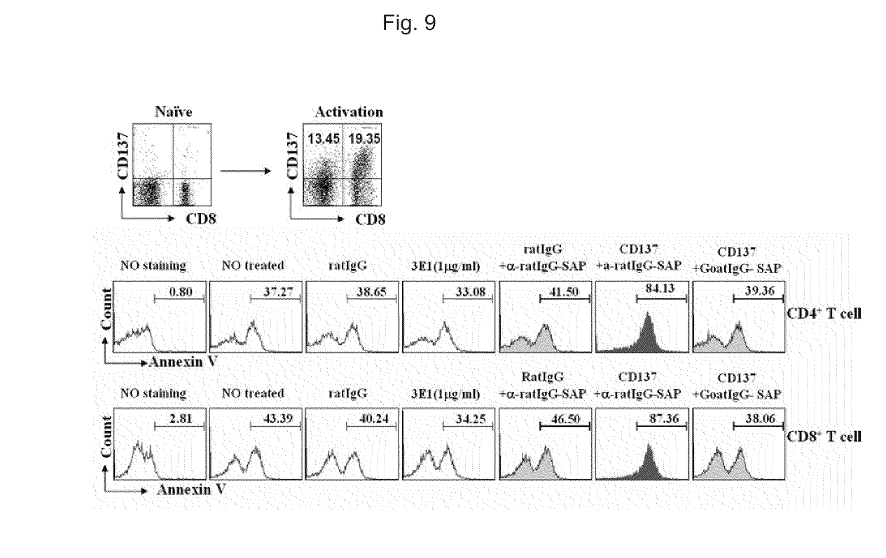

In general, the anti-CD137 antibody is known to induce cell proliferation and differentiation when it binds to CD137 in activated CD4⁺ and CD8⁺ T cells. Hence, the present inventors investigated whether the use of the anti-CD137 antibody-doxorubicin complex of the present invention could suppress the proliferation the CD137 positive cells and induce apoptosis them. In accordance with one embodiment of the present invention, the anti-CD137 antibody-doxorubicin complex was treated by using, as the CD137 positive cells, a CD137 expressing mouse cell lines and activated CD4⁺ and CD8⁺ cells of the spleen and lymph nodes and the level of apoptosis was measured. As a result of the measurement, it was seen that the higher the concentration of the treated complex, the more the level of apoptosis of immune cells from mouse spleen, whereas genetically CD137-deficient cells were not killed (see FIGS. 4 and 5a). Also, the same result was obtained in a complex of an anti-CD137 antibody and saporin as a toxin (see FIGS. 8 and 9).

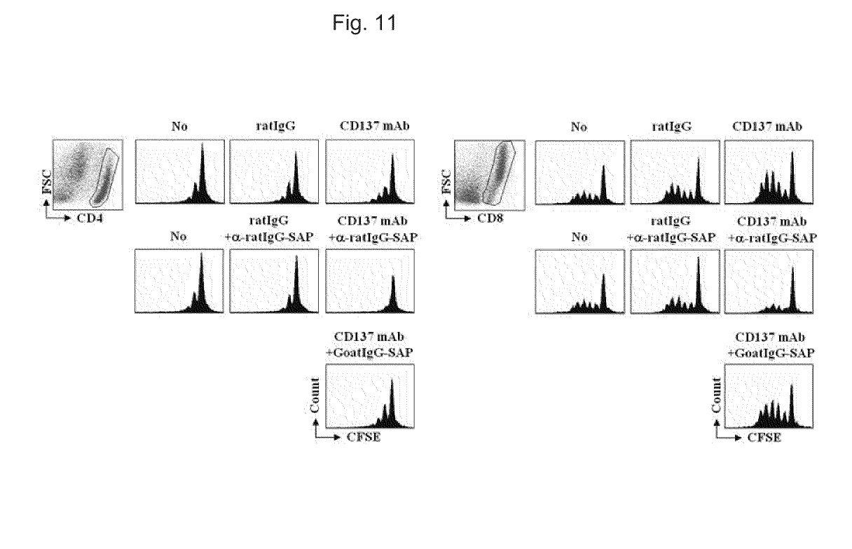

In another embodiment of the present invention, an investigation was made of the effect of the anti-CD137 antibody-doxorubicin complex of the present invention on CD137 positive cells. That is, the anti-CD137 antibody, the anti-CD137 antibody-doxorubicin complex, and doxorubicin alone were respectively treated in activated immune cells isolated from the mouse spleen and the proliferation of the cells was observed. As a result of the observation, if the anti-CD137 antibody alone was treated, cell proliferation was induced, whereas if the complex was treated, cell proliferation was suppressed to a significant extent (see FIGS. 5b and 5c). Also, the same result obtained in the complex of the anti-CD137 antibody and saporin as the toxin (see FIG. 11).

Therefore, the present invention can provide a complex of an anti-CD137 antibody and a toxin which can suppress the activation of CD137 positive cells.

Moreover, the anti-CD137 antibody-toxin complex in accordance with the present invention is characterized in that it can selectively deplete CD137 expressing cells, i.e., CD137 positive cells, and suppress their proliferation irrespective of the type of an antigen.

Generally, the term “antigen” refers to a substance that induces an immune response, and the immune response includes production of antibodies and stimulation of activated cells. The antigen is reactive with an antibody or an activated cell receptor. In the present invention, the CD137 expressing cells can be activated by an alloantigen, a heterologous antigen, or a foreign antigen.

As used herein, the term “alloantigen” refers to an antigenic substance derived from an individual with different genetic factors of the same species, and the term “heterologous antigen” refers to an antigenic substance derived from a species with different genetic factors.

Further, diseases mediated by the activation of the CD137 positive cells may include diseases that may be caused by immune responses to the CD137 positive cells, and the types of such diseases may include, but not limited to, autoimmune diseases, graft versus host diseases, transplantation, cancer, and inflammatory diseases.

In general, autoimmune diseases are characterized in that an antibody reacting against host tissues are autoreactive to endogenous self-peptides to generate immune effector T cells. An immune response of the T cells causes damage to the cells or tissues and thus induces autoimmune diseases. The types of the autoimmune diseases may include, but not limited to, Crohn disease, rheumatoid arthritis, osteoarthritis, reactive arthritis, psoriatic arthritis, hay fever, atopy, multiple sclerosis, Sjogren's syndrome, sarcoidosis, insulin-dependent diabetes mellitus, autoimmune thyroiditis, ankylosing spondylitis, and scleroderma.

Graft versus host disease (GVHD) commonly develops in various symptoms in allogeneic stem cell transplant recipients, and is often accompanied by other clinical complications such as diseases like fibrosis and scleroderma (Gilliam A C, J. Invest., Dermatol., 123, 251-257, 2003). It is known that the GVHD is mediated by a pathogenic donor T cell produced after alloreactivity to a minor histocompatibility (mH) antigen or autoantigen against a host, wherein the T cell attacks a target tissue by stimulating the secretion of infectious and fibrous cytokines, or production of autoantibodies, in addition to direct cytolytic attack.

Moreover, for successful organic transplantation, it is necessary to overcome immune rejection in a recipient of cells and organs to be transplanted. When a transplantation Is performed, the important mediators of immune rejection are T cells. An immune response is induced through recognition by the T cell receptor of the major histocompatibility complex (MHC) expressed on grafts, whereby transplant rejection occurs. Although the success rate of transplantation has risen recently with the improvement of surgical procedures and HLA typing techniques and the development of immunosuppressive agents, the death rate due to immune rejection and the side effects of the immunosuppressive agents is still high. Thus, there is a demand for the development of a novel effective and safe immunosuppressive agent.

Further, the types of cancers mediated by the activation of the CD137 positive cells may include, but not limited to, blood cancer, cervical cancer, lung cancer, pancreatic cancer, non-small cell lung cancer, liver cancer, colon cancer, skin cancer, head or neck cancer, skin or intraocular melanoma, uterine cancer, ovarian cancer, colorectal cancer, stomach cancer, cancer near the anus, breast cancer, oviduct carcinoma, endometrial carcinoma, vaginal carcinoma, esophagus cancer, small intestinal cancer, endocrine gland cancer, thyroid cancer, parathyroid cancer, adrenal cancer, prostate cancer, bladder cancer, and kidney cancer.

In addition, the types of infectious diseases mediated by the activation of the CD137 positive cells may include, but not limited to, asthma, tenosynovitis, food allergy, systemic lupus erythematosus, vasculitis, dermatitis, contact dermatitis, and sepsis.

Therefore, a substance or method for suppressing the activation of the CD137 positive cells can prevent or treat the aforementioned diseases caused by the activation of the CD137 positive cells, and the anti-CD137 antibody-toxin complex according to the present invention can prevent or treat the diseases mediated by the activation of the CD137 positive cells because it shows excellent effects in depleting the CD137 positive cells by promoting the apoptosis of the CD137 positive cells or suppressing their proliferation.

Accordingly, the present invention can provide a pharmaceutical composition comprising, as an effective component, a complex of an anti-CD137 antibody and a toxin, which is capable of preventing or treating diseases mediated by the activation of the CD137 positive cells.

The composition in accordance with the present invention may comprise a pharmaceutically effective amount of the anti-CD137 antibody-toxin complex alone or together with at least one pharmaceutically acceptable carrier, excipient, or diluent. As used herein, the term “pharmaceutically effective amount” refers to an amount sufficient to prevent, reduce, and treat the symptoms of a disease mediated by the activation of the CD137 positive cells.

The pharmaceutically effective amount of the anti-CD137 antibody-toxin complex in accordance with the present invention is 0.5 to 100 mg/day/kg body weight, and preferably, 0.5 to 5 mg/day/kg bodyweight. The pharmaceutically effective amount may be suitably varied depending on disease and its severity, the age, bodyweight, medical condition and sex of a patient, an administration route and treatment period.

As used herein, the term “pharmaceutically acceptable” refers to a composition which is physiologically acceptable and, when administered to the human beings, does not cause allergic reactions such as gastrointestinal disorders, dizziness, or similar responses. Examples of the carrier, excipient or diluent may include lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. The pharmaceutical composition may additionally comprise fillers, anticoagulants, lubricants, wetting agents, fragrances, emulsifiers, preservatives, etc.

Also, the inventive pharmaceutical composition can be formulated using a method known in the art so as to provide quick, sustained or delayed release of the active ingredient after administration to mammals. The composition may be in the form of powder, granules, tablets, emulsion, syrup, aerosol, soft or hard gelatin capsules, sterilized injection solution, or sterilized powder.

The composition in accordance with the present invention can be administered through various routes, including oral, transdermal, subcutaneous, intravenous and intramuscular routes. The dosage of the active ingredient can be suitably selected depending on various factors, including an administration route and the age, sex, bodyweight and disease severity of a patient. The composition of the present invention may be administered in combination with a well-known compound having the effect of preventing, reducing, or treating the symptoms of a disease mediated by the activation of the CD137 positive cells.

Moreover, the present invention can provide a method for depletion of CD137 positive cells in vitro, comprising the step of contacting an anti-CD137 antibody-toxin complex with the CD137 positive cells, which can effectively prevent and treat diseases caused by the activation of CD137 expressing cells, and furthermore provide a method for selective depletion of CD137 positive cells.

Further, in the present invention, the CD137 positive cells are cytotoxic cells, that is, activated cells expressing CD137, and the CD137 positive cells may be selected, but not limited to, from the group consisting of T cells, B-cells, dendritic cells, natural killer (NK) cells, macrophages, cancer cells, and myeloid cells containing neutrophils, basophils, and eosinophils. In one embodiment of the present invention, CD4⁺ cells, CD8⁺ T cells, and T-helper cells belonging to T cells are used as the CD137 positive cells.

In addition, when the anti-CD137 antibody-toxin complex is contacted with the CD137 positive cells, the CD137 positive cells may be treated with the anti-CD137 antibody-toxin complex at a concentration of 0.1 to 5.0 μg/ml.

Additionally, the present invention can provide a method for the treatment or prevention of diseases mediated by the activation of CD137 positive cells, the method comprising the step of administering an anti-CD137 antibody-toxin complex or a composition comprising the complex to an individual requiring the same. The individual may be any animal except a human.

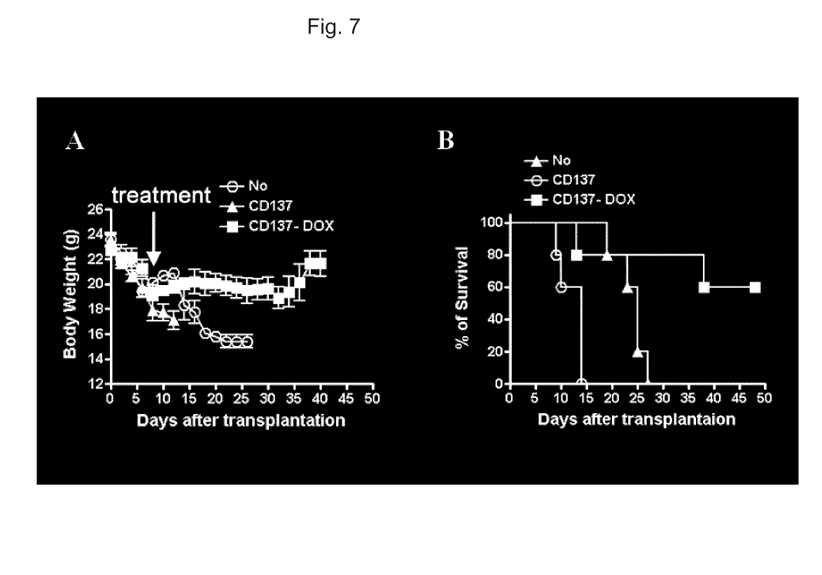

In one embodiment of the present invention, an acute graft-versus-host disease (GVHD) model was used as an animal experimental model. Acute GVHD is well known to be a disease mediated by donor immune cells, i.e., activated T cells. Thus, it was investigated whether the complex in accordance with the present invention suppresses the activation of the T cells or induces apoptosis, and as a result, it was observed that, when an anti-CD137 antibody-doxorubicin complex was intraperitoneally injected to a mouse model that expresses CD137 by inducing acute GVHD, the recovery and survival rate of the mouse model increased after administration (see FIGS. 7a and 7b).

Therefore, the present inventors found out that the anti-CD137 antibody-toxin complex in accordance with the present invention was very effective in the treatment of a disease mediated by the activation of the CD137 positive cells.

Consequently, the anti-CD137 antibody-toxin complex of the present invention or a composition comprising the complex can be administered to mammals except humans in the same manner as the above-described administration of a composition. For example, it can be administered through oral, rectal, intravenous, intramuscular, hypodermic, intrauterine, epidural or intracerebroventricular injections.

Now, the present invention will be described in detail with reference to Examples. However, these examples are only to illustrate the present invention, and it is not construed that the scope of the present invention is limited by the examples.

### EXAMPLE 1

### Determination of Endocytosis of CD137 And Anti-CD137 Antibody

First, the present inventors conducted the following experiment in order to determine whether or not an anti-CD137 antibody is effective in delivering a toxin, i.e., a cytotoxic drug, to target cells.

### <1-1>Determination of Internalization of Andti-CD137 Antibody Into Cell Lines

First, male mice (57BU6, BDF1) of 10 weeks of age purchased from Charles River Orient were used as experimental animals in the present invention, and were bred in a SPF (specific pathogens free) facility of Biomedical Research Center, Ulsan University. Also, an anti-mouse CD137 monoclonal antibody used in the following experiments was isolated and purified from ascites by a protein G column (Sigma-Aldrich, St. Louis, Mo.), the ascites being collected after the administration of hybridoma cells (3E1 and 3H3), a gift from Dr. Robert Mittler, Emory University, to nude mice, and then was purified. An anti-human CD137 monoclonal antibody (4B4, 4785) was isolated and purified from ascites collected from Balb/c in the same manner as the mice. Control rat IgG was purchased from Sigma-Aldrich Korea.

In an experiment for the determination of internalization of CD137 into cell lines, first, CD137 transfected EL-4 cell line and CTLL-R8 cell line which expressed CD137 on its surface were collected and washed twice with PBS. Cells were harvested and stained with PE-fluorescence-labeled anti-CD137 antibodies at 4° C. for 30 minutes. After that, the cell were washed three times with PBS to remove PE-anti-CD137 antibodies not bound to CD137, and suspended in cell culture fluid (RPMI1640 with 10% FBS and antibiotic) for 4 hours. After that, the cells were attached to a poly-L-lysine-coated slide and fixed with a mounting solution (Fluoromount G; Southern Biotech), and the intracellular location of the PE-anti-CD137 antibodies was determined with a fluorescence microscope (Olympus FV500).

### <1-2>Determination of Internalization of CD137 And Anti-CD137 Antibody Complex Into Mouse Cell Lines And T cells

To determine the internalization of a CD137 and anti-CD137 antibody complex, immune cells were isolated from spleen and lymph nodes. The isolated immune cells were counted, and 5×10⁶/ml cells were suspended in 10 ml of cell culture fluid (RPMI1640 with 10% FBS and antibiotic) and cultured with anti-mouse CD3 mAb at a concentration of 0.2 μg/ml for 24 hours. After 24 hours, the cells were collected and washed twice with PBS. Then, a part of the cells was harvested and stained simultaneously with PE-fluorescence-labeled anti-CD137 mAb-PE, FITC-fluorescence labeled anti-CD4 mAb-FITC or FITC-fluorescence-labeled anti-CD8 mAb-FITC, whereby CD137 expression on CD4⁺ T cells and CD8⁺ T cells was detected. After CD137 expression was detected, CD4⁺ T cells and CD8⁺ T cells were isolated in pure form from the cultured immune cells using MACS method. PE-fluorescence-labeled anti-CD137 antibodies were bound to the isolated CD4⁺ T cells and CD8⁺ T cells at 0.4 μg/ml under 4° C. for 30 minutes and washed three times with PBS to remove PE-anti-CD137 antibodies not bound to CD137,and then suspended in cell culture fluid (RPMI1640 (without 10% FBS) and antibiotic) for 4 hours. After 4 hours of culturing, the cells were collected, stained with an FITC-fluorescence-labeled anti-CD8-mAb under 4° C. for 30 minutes, and washed three times with PBS. After the washing, the cells were fixed for 15 minutes with 4% paraformaldehydem, washed three times with PBC, and permeabilized in 0.25% Triton X100. After that, the cells were stained for 1 hour with FITC-fluorescence-labeled anti-EEA-1 antibodies. After the staining, the cells were washed three times with PBS and attached to a poly-L-lysine-coated slide and fixed with a mounting solution (Fluoromount G; Southern Biotech), and the intracellular location of the PE-anti-CD137 antibodies was determined with a fluorescence microscope (Olympus FV500).

As a result, as shown in FIG. 1, it was demonstrated that the PE-anti-CD137 antibodies on the cell surface at incubation time 0 were internalized into the cell lines and T cells (CD4⁺ T cells and CD8⁺ T cells) after 4 hours of incubation. Also, in order to determine whether such internalization was induced by endocytosis, the cells were simultaneously stained with an endocytosis marker EEA1. As a result, it was found that the CD137 molecular, and the CD137 and anti-CD137 antibody complex were internalized into the cells by endocytosis

### <1-3>Determination of Internalization of CD137 And Anti-CD137 Antibody Complex Into Human And Primate T cells

Peripheral Blood Mononuclear cells (PBMC) were isolated from human peripheral blood and monkey peripheral blood (Center for animal resource development college of medicine, SEOUL national university) by using a Ficoll-Paque™ Plus (GE Healthcare Biosciences, Uppsala, Sweden). The isolated PBMC were counted and adjusted to 5×10⁶/ml, suspended in 10 ml of cell culture fluid (RPMI1640 with 10% FBS and antibiotic), and cultured in a cell culture medium with anti-CD3 mAb (human clone: OKT-3, monkey clone: FN-18; U-cytech bioscience, Netherlands) at a concentration of 0.2 μg/ml for 24 hours. After the culturing, CD137 expression was detected. After CD137 expression was detected, the cultured cells were washed twice, and reacted with and bound to a PE-fluorescence-labeled anti-human-CD137 antibodies under 4° C. for 30 minutes. After that, the cells were washed three times with PBS to remove PE-anti-human CD137 antibodies not bound to CD137, and then suspended in cell culture fluid (RPMI1640 with 10% FBS and antibiotic) for 4 hours. After 4 hours of culturing, the cells were collected again, stained with an FITC-fluorescence-labeled anti-CD8-mAb (clone:) under 4° C. for 30 minutes, and washed three times with PBS. After the washing, the cells were fixed for 15 minutes with 4% paraformaldehydem, washed three times with PBC, and permeabilized in 0.25% Triton X100. After that, the cells were stained for 1 hour with FITC-fluorescence-labeled anti-EEA-1 antibodies. After the staining, the cells were washed three times with PBS and attached to a poly-L-lysine-coated slide and fixed with a mounting solution (Fluoromount G; Southern Biotech), and the intracellular location of the PE-anti-CD137 antibodies was determined with a fluorescence microscope (Olympus FV500).

As a result, as shown in FIGS. 2a to 2c, it was confirmed that, when PBMC isolated from a human and a monkey were cultured with the anti-CD3 antibodies, CD137 was expressed on T cells (CD4⁺ T cells and CD8⁺ T cells) (see FIG. 2a). It was also confirmed that, when the anti-CD137 antibodies were cultured with CD137 of activated T cells, the CD137 and anti-CD137 antibody complex was internalized into the cells in the same manner as the mice (see FIGS. 2b and 2c).

Consequently, based on the above results, the present inventors found out that the anti-CD137 antibody used in the present invention is very suitable as a carrier material for delivering toxins into CD137 positive cells and depleting the cells. They also found out that the anti-CD137 antibody could be used for monkey and human cells, as well as for mice, to deliver toxins.

### EXAMPLE 2

### Synthesis of Anti-CD137 Antibody-Doxorubicin Complex

By confirming, through the experiment of Example 1, that an anti-CD137 antibody was internalized into cells by binding to CD137, a toxin to be delivered to CD137 positive cells was selected to synthesize a complex of the toxin and the anti-CD137 antibody. Doxorubicin, a kind of antitumor agent, was selected as the toxin, and a complex of an anti-CD137 antibody (clone: 3H3, 3E1) and doxorubicin was prepared by Peptron (Daejeon, Korea). First, MPBH and doxorubicin were added at a ratio of 1:10 to DMSO containing sodium sulfate, reacted under 50° C. for 30 minutes, and centrifugated to remove the sodium sulfate. After that, precipitates were produced by ether, and freeze-dried to obtain activated doxorubicin. Next, the anti-CD137 antibody was reduced to bind the activated doxorubicin to the anti-CD137 antibody. That is, 16 mg of anti-CD137 antibody in 1 ml of 40 mM DTT was partially reduced with 0.1M sodium phosphate containing 5 mM EDTA for 40 minutes under 37° C. After that, the anti-CD137 antibody was desalted with a 50 mM ABS (acetate buffered saline) solution (pH 5.3) containing 2 mM EDTA, and then the amount of free thiol groups was measured by Ellman's test. Next, 15 mg of anti-CD137 antibody was dissolved in 1.5 ml of acetate buffer, 2 mg of doxorubicin was dissolved in 500 ul DMSO, and the two dissolved solutions were mixed together and adjusted to pH 7.2 under ice condition. After that, the mixture was reacted in ice for 2 hours, and desalted with a PBS solution, thereby preparing a complex of the cysteine of the partially reduced anti-CD137 antibody and doxorubicin. The complex was isolated in pure form by FPLC, and it was determined whether the prepared complex normally binds to CD137 molecules. To this end, the complex was labeled with FITC fluorescence (CD137-doxorubicin-FITC), and the binding strength of the complex to CD137 was compared with that of the anti-CD137 antibody (CD137-FITC) to CD137.

As a result, as shown in FIG. 3a, it was confirmed that the anti-CD137 antibody-doxorubicin complex was isolated in pure form by FPLC. As a result of the measurement of the binding strength of the complex to CD137, as shown in FIG. 3b, it was confirmed that, even when doxorubicin was conjugated to the anti-CD137 antibody, the complex was normally bound to CD137.

### EXAMPLE 3

### Measurement of Activation of Cell Apoptosis And Proliferation In Vitro By Anti-CD137 Antibody-Doxorubicin Complex

### <3-1>Measurement of Activation of Cell Apoptosis By Anti-CD137 Antibody-Doxorubicin Complex

Lymphocytes isolated from spleen and lymph nodes of normal mice and CD137-depleted mice were treated with an anti-CD3 antibody at a concentration of 0.2 μg/ml and cultured for 24 hours in cell culture fluid. After that, the cultured cells were collected and washed twice with PBS, and a small amount of cells (1×10⁵ cells) were harvested and fluorescence-stained with PE-anti-CD137 mAb and FITC-anti-CD8 mAb-FITC or FITC-anti-CD4 mAb under 4° C. for 30 minutes. After being stained, the cells were washed twice with PBS, and CD137 expression on CD4⁺ T cells and CD8⁺ T cells was detected by a flow cytometry (FACS caliber, BD). When CD137 expression was detected, the cultured cells (1×10⁶ cells) were treated at each concentration with the anti-CD137 antibody and doxorubicin prepared in Example 3 of the present invention and with doxorubicin alone as a control group, and reacted under 4° C. for 30 minutes. After the reaction, the cells were washed three times with PBS to remove any unbound anti-CD137 antibody-doxorubicin complex. After the washing, the cells were suspended in 0.5 ml of cell culture fluid, and additionally cultured for 48 hours on 48-well cell culture plates. After the culturing, the cells were collected and stained with Annexin V for 20 minutes, and the percentage of Annexin V positive cells were analyzed by a flow cytometry. Also, to reveal a direction association between the anti-CD137 antibody-doxorubicin complex in accordance with the present invention and cell apoptosis, CD137 was expressed on mouse T cells in the same manner as above, and the cells were treated with FITC-fluorescence labeled anti-CD137 antibody and doxorubicin and cultured for 24 hours, and then stained with PE-Annexin V, followed by the analysis of the relationship between the fluorescent locations of the anti-CD137 antibody-doxorubicin complex and the Annexin V by a flow cytometry.

As shown in FIG. 4, as a result of determination of the efficacy of apoptosis by the anti-CD137 antibody-doxorubicin complex of the present invention was determined on mouse cell lines and spleen immune cells, it was demonstrated that apoptosis by the anti-CD137 antibody-doxorubicin complex was concentration-dependently increased in CD137 expressing CTLL-R8 (see FIG. 4b). Also, as a result of measurement of the effect of apoptosis by treating CD137 expressing mouse immune cells with the anti-CD137 antibody-doxorubicin complex, it was demonstrated that the T cells (CD4⁺ T cells and CD8⁺ T cells) were killed depending on the concentration of the complex of the present invention (see FIG. 5a). On the contrary, the efficacy of such apoptosis was not observed on the spleen immune cells of the mice genetically deficient in CD137.

Accordingly, it was found that intracellular delivery of doxorubicin using the anti-CD137 antibody in accordance with the present invention occurs specifically to CD137, and further that a doxorubicin-conjugated anti-CD137 antibody was effective in selective depletion of CD137 positive cells.

### <3-2>Measurement of Activation of Cell Proliferation of Anti-CD137 Antibody-Doxorubicin Complex

Lymphocytes (stimulated with anti-CD3 mAb) expressing CD137 were counted and 2×10⁵/well cells were dispensed on a 96 well culture plate, and treated with an anti-CD137 antibody, an anti-CD137 antibody-doxorubicin complex, and doxorubicin, respectively, at a concentration of 5 μg/ml and then cultured for 48 hours. When the incubation time reaches 40 hours, each well was treated with 1 uCi of thymidine (3H) labeled with radioactive isotope. After 48 hours of culturing, the amounts of isotope in the cultured cells for each experimental group were compared with each other by a micro beta counter.

As a result, it was demonstrated that, if the cells were treated with the anti-CD137 antibody alone, this induces the proliferation of immune cells, as is known that the anti-CD137 antibody generally binds to CD137 of activated CD4⁺ T cells and CD8⁺ T cells and induces the proliferation and differentiation of the cells. On the contrary, it was demonstrated that the anti-CD137 antibody-doxorubicin complex of the present invention suppressed the proliferation of immune cells by the anti-CD137 antibody to a significant extent (see FIG. 5b). Also, it was determined whether or not the doxorubicin-conjugated anti-CD137 antibody was actually bound to CD 137 positive cells and induces apoptosis. As shown in FIG. 5c, it was demonstrated that the anti-CD137 antibody-doxorubicin complex were present as positive on the Annexin V positive cells of the CD8⁺ T cells. Accordingly, from the above results, the present inventors found that the anti-CD137 antibody-doxorubicin complex did not induce cell proliferation but was active for inducing cell apoptosis, and that the doxorubicin-conjugated anti-CD137 antibody induced cell apoptosis selectively on CD137 positive cells.

### EXAMPLE 4

### Determination of Efficacy of Anti-CD137 Antibody-Doxorubicin Complex In Vivo

As it was confirmed, by Example 3, that the anti-CD137 antibody-doxorubicin complex in accordance with the present invention is active in inducing apoptosis selectively on CD137 positive cells in vitro and suppressing cell proliferation, the present inventors examined whether or not the above activation was performed in vivo as well. As an experimental model for the examination, acute graft-versus-host disease (GVHD) mice were used. Acute GVHD is generally known to be a disease mediated by donor immune cells, i.e., activated T cells. Specifically, for the in vivo experiment, first, BDF1 mice were used as recipient mice in order to induce acute GVHD, and C57BL/6 mice were used as donor mice. After that, the BDF1 mice were irradiated at 750 rads, and marrow cells (5×10⁶cell/mouse) of the donor mice and lymphocytes (2.5×10⁷/mouse) isolated from the spleen of the donor mice were injected into the irradiated mice through the tail veins. After the cell injection, the mice were sacrificed every other day to harvest spleens, lymph nodes, and blood, and the immune cells were isolated from each of the collected samples and stained simultaneously with FITC-anti-CD4 mAb+PE-anti-CD137 mAb or FITC-anti-CD8 mAb+PE-anti-CD137 mAb, whereby CD137 expression on CD4⁺ T cells and CD8⁺ T cells was detected by a flow cytometry. Also, as for the administration of the anti-CD137 antibody-doxorubicin complex prepared in the present invention, the complex was intraperitoneally injected at 100 μg/mouse 7 days after induction of acute GVHD, and a group administered with the anti-CD137 antibody alone and a group administered with nothing were used as control groups. To estimate the development of disease after induction of acute GVHD, body weight changes and survival of the mice were monitored every day.

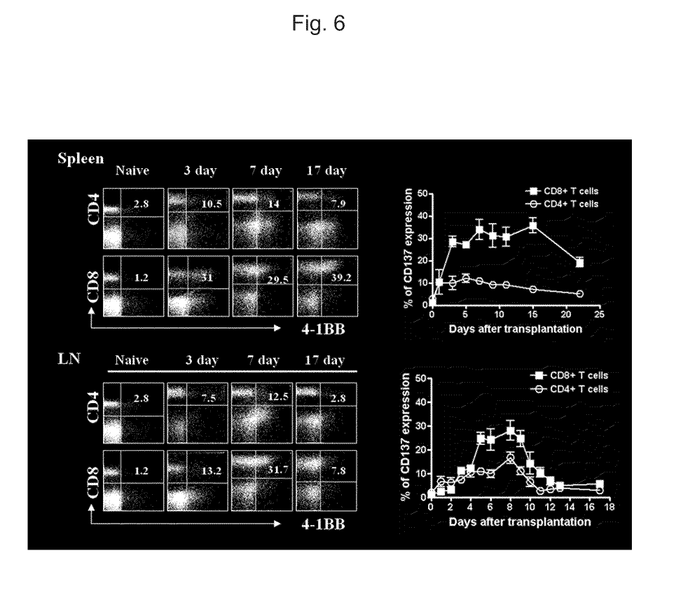

As a result of investigation of expression of CD137 molecules in the T cells after induction of acute GVHD, as shown in FIG. 6, it was confirmed that CD137 was expressed by the induction of acute GVHD, and it was demonstrated that the level of expression of the CD4⁺ T cells and CD8⁺ T cells of the lymph nodes reached its peak 7 to 8 days after the induction of the disease. On the other hand, the level of expression of the spleen T cells was demonstrated to reach its peak 7 to 8 days after the induction of the disease and remain there. From this result, the present inventors could predict that CD137 positive cells could be depleted by the anti-CD137 antibody in vivo.

Moreover, based on the pattern of expression of CD137 caused by acute GVHD, the anti-CD137 antibody-doxorubicin complex was injected intraperitoneally 7 days after the peak of CD137 expression to detect treatment effects. As indices for treatment effects, weight changes and survival were monitored. As a result, as shown in FIG. 7, it was confirmed that the control group administered with nothing and the control group administered with the anti-CD137 antibody alone showed significant decrease in body weight and survival, whereas the group administered with the anti-CD137 antibody-doxorubicin complex showed recovery of body weight and increase in survival after the administration (see FIGS. 7a and 7b). Therefore, from this result, the present inventors found that the anti-CD137 antibody-doxorubicin complex could effectively treat acute GVHD, and further, that the complex of the present invention could deplete CD137 positive T cells in vivo as well as in vitro and thus was useful in treating a specific disease.

### EXAMPLE 5

### Synthesis of Anti-CD137 Antibody-Saporin Complex

Saporin, as well as doxorubicin, was used as a toxin that binds to an anti-CD137 antibody to synthesize a complex of the anti-CD137 antibody and saporin. Synthesis of the complex with saporin was carried out by binding saporin conjugated to various types of secondary antibodies to a primary anti-CD137 antibody. Saporin conjugated to the secondary antibodies was purchased from Advanced Target Systems, Inc. Also, in the following experiment, anti-rat IgG-saporin was used in a mouse experiment, and anti-mouse IgG-saporin was used in a human experiment. As the IgG type of the anti-CD137 antibody used for mice is rat IgG, anti-rat IgG-saporin can be conjugated to rat IgG of the antibody. On the other hand, as the IgG type of the anti-CD137 antibody used for humans was mouse IgG, anti-mouse IgG-saporin can be conjugated to mouse IgG of the anti-CD137 antibody. In this embodiment, the anti-CD137 antibody-saporin complex was prepared by binding saporin conjugated to a secondary antibody to a primary anti-CD137 antibody, and the complex was used in the following examples. As for the preparation of the antibody-saporin complex, saporin was dissolved in 50 mM sodium borate buffer (pH 9.0) and reacted with 2-iminothiolane for 60 minutes at a final concentration of 1 mM. After the reaction, saporin containing a sulfhydryl group was removed by gel filtration on a Sephadex G25 column, and the removed saporin was reduced with 20 mL 2-mercaptoethanol and filtered on a Sephadex G25 column to remove the reduced saporin. The removed saporin and the antibody were mixed at a 10:1 molar ratio and reacted at room temperature for 16 hours, were subjected to gel filtration on a Sephacryl S200 high-resolution column, and equilibrated with phosphate buffer saline (PBS, pH 7.4) to elute an antibody-saporin complex. (Bolognesi, A. et al., In Vitro anti-tumor activity of anti-CD80 and anti-CD86 immunotoxins containing type 1 ribosome-inactivating proteins. Br J Haematol, Aug. 2000. 110 (2): p. 351-61)

### EXAMPLE 6

### Measurement of Activation of Cell Apoptosis By Anti-CD137 Antibody-Saporin Complex

### <6-1>Measurement of Activation of Apoptosis In Cell Lines

EL-4 cell lines (5×10⁵ cells) transfected with CD137 were treated with rat IgG, anti-CD137 antibody, rat IgG+anti-rat IgG-saporin, and anti-CD137 antibody+anti-rat IgG-saporin at a concentration of 1 μg/ml and cultured for 24 hours and 48 hours, respectively. Next, the cells were collected and stained with FITC-fluorescence labeled Annexin V to measure cell apoptosis in each experimental group by a flow cytometry.

As a result, as shown in FIG. 8, it was demonstrated that no cell apoptosis was observed in a control group administered with nothing, whereas there was an increase in cell apoptosis to a significant extent in a group treated with anti-CD137 antibody and anti-rat IgG-saporin. Therefore, from this result, it was found that the saporin used in the present invention was suitable for use as a substance for selectively depleting cells and increasing cell toxicity.

### <6-2>Activation of Apoptosis In Mouse Cells By Anti-CD137 Antibody-Saporin Complex

Immune cells isolated from the spleen and lymph nodes of mice were treated with an anti-CD3 antibody at a concentration of 0.2 μg/ml and cultured in cell culture fluid for 24 hours. The cultured cells were collected and washed twice with PBS, and a small amount of cells (1×10⁵ cells) were harvested and fluorescence-stained with PE-anti-CD137 mAb and FITC-anti-CD8 mAb-FITC or FITC-anti-CD4 mAb under 4° C. for 30 minutes. After being stained, the cells were washed twice with PBS, and CD137 expression on CD4⁺ T cells and CD8⁺ T cells was detected by a flow cytometry (FACS caliber, BD). When CD137 expression was detected, the cultured cells (5×10⁵ cells) were treated with rat IgG, anti-CD137 antibody, rat IgG+anti-rat IgG-saporin, and anti-cD137 antibody+anti-rat IG-saporin, respectively, at a concentration of 1 μg/ml and cultured for 48 hours in a 48 well cell culture plate. After the culturing, the cells were collected, washed twice with PBS, and stained with PE-Cy5-anti-CD4 mAb, PE-anti-CD8 antibody, and FITC-Annexin V to analyze the positive rate of Annexin V in the CD4+ T cells and CD8+ T cells by a flow cytometry.

As a result, it was confirmed that, when the immune cells isolated from the spleen were stimulated and activated with the anti-CD3 antibody, CD137 molecules were expressed on the T cells, and it was demonstrated that, if activated T cells were treated with the respective antibodies, the group treated with the anti-CD137 antibody and the anti-rat IgG-saporin showed cell apoptosis to a significant extent in comparison with control groups (see FIG. 9). Therefore, from this result, the present inventors found out that the intracellular delivery of saporin via the anti-CD137 antibody in accordance with the present invention was effective in selectively depleting CD137 positive cells.

### <6-3>Activation of Cell Apoptosis In Human Cells By Anti-CD137 Antibody-Saporin Complex

PBMC isolated from human peripheral blood were treated with an anti-CD3 antibody at a concentration of 0.2 μg/ml and cultured in cell culture fluid for 24 hours. The cultured cells were collected and washed twice with PBS, and a small amount of cells (1×10⁵ cells) were harvested and fluorescence-stained with PE-anti-CD137 mAb and FITC-anti-CD8 mAb-FITC or FITC-anti-CD4 mAb under 4° C. for 30 minutes. After being stained, the cells were washed twice with PBS, and CD137 expression on CD4⁺ T cells and CD8⁺ T cells was detected by a flow cytometry (FACS caliber, BD). When CD137 expression was detected, the cultured cells (5×10⁵ cells) were treated with 4B4 (agonist antibody), 4785 (antagonist antibody), 4B4+anti-mouse IgG-saporin, and 4785+anti-mouse IgG-saporin, respectively, at a concentration of 1 μg/ml and cultured for 48 hours in a 48 well cell culture plate. After the culturing, the cultured cells were collected, washed twice with PBS, and stained with Annexin V to analyze the positive rate of Annexin V by a flow cytometry.

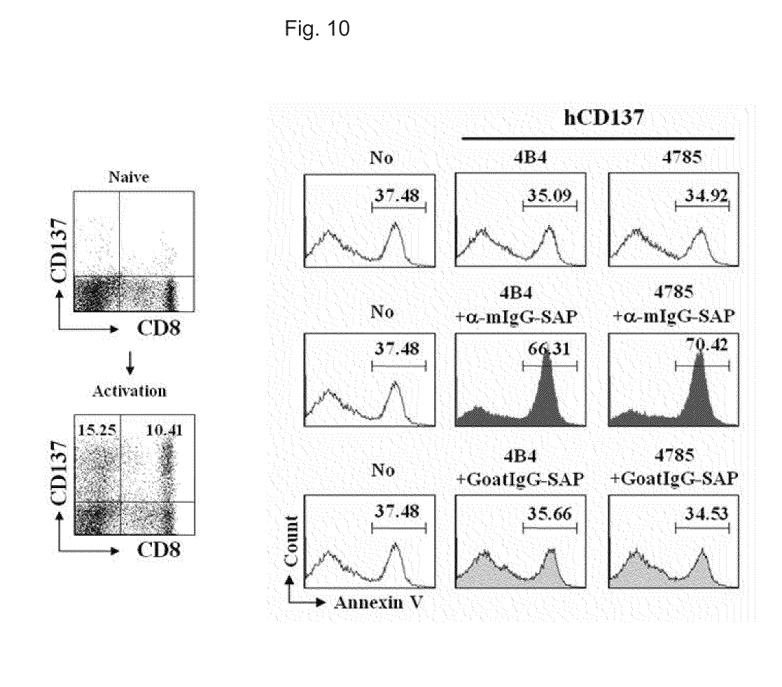

As a result, as shown in FIG. 10, it was confirmed that cell apoptosis occurred to a significant extent in both of the agonistic antibody-clone (4B4) and the antagonistic antibody-clone (4785). From this result, the present inventors found out that the anti-CD137 antibody-saporin was effective in the depletion of CD137 positive cells on human immune cells as well as on mouse immune cells.

### EXAMPLE 7

### Measurement of Activation of Suppression of Cell Division of Alloantigen Specific T Cells By Anti-CD137 Antibody-Saporin Complex

It was confirmed through the previous examples that the anti-CD137 antibody-toxin complex in accordance with the present invention induced cell apoptosis by selectively binding to CD137 positive T cells and delivering a toxin. Therefore, the present inventors predicted that the anti-CD137 antibody-toxin complex in accordance with the present invention could deplete CD137 positive cells when CD137 expression was activated irrespective of the type of antigen, and thus measured the activation of suppression of cell division of alloantigen specific T cells by the anti-CD137 antibody-saporin complex. Samples and cells prepared for the measurement were treated as follows.

### (1) Isolation And Preparation of Antigen Presenting Cells (APC)

Immune cells isolated from mouse spleen and human blood (50 ml) were cultured in cell culture fluid for 24 hours, and then floating cells were removed and the plate was washed twice with PBS. After washing, the plate was treated with trypsin-EDTA, and adhered cells were collected, washed again twice with PBS, resuspended in 2 ml of PBS, and then transferred to a 15 ml tube. After that, the prepared cells were put in a irradiator and irradiated with 3000 rads. After the irradiation, the cells were washed once with cell culture fluid and the number of the cells were counted and adjusted to 1×10⁶ cells/ml for use in the experiment.

### (2) CFSE Labeling

Immune cells isolated from mouse spleen and human blood were counted, and 1×107 cells were suspended in 7 ml of PBS, treated with 0.25 μg of CFSE (molecular probe), and cultured under 37° C. for 5 minutes while protected from light. After 5 minutes of the culturing, the cells were treated with 3 ml of FBS, cultured for 30 seconds, and washed three times with PBS for use in the experiment.

### (3) In Vitro MLR

The antigen presenting cells (1×10⁵ cells) prepared by the above method and the CFSE-labeled immune cells (2×10⁵ cells) were mixed at a ratio of 1:2 and treated with 0.2 μg/ml of anti-CD3 antibody, and cultured on 48-well culture plates. The culture cells were collected and CD137 expression was detected. The cells were treated with respective antibodies, anti-rat IgG-saporin, and anti-mouse IgG-saporin, respectively, at 1 μg/ml, cultured for 48 hours, collected after the 48 hours of culturing, and washed twice with PBS. After the washing, the cells were floated in a flow cytometric analysis solution (2% BSA-PBS) and stained with PE-Cy5-anti-CD4 antibody and PE-anti-CD8 antibody to analyze the fluorescence of CFSE in CD4+ T cells and CD8+ T cells by a flow cytometry (FACS).

As a result of measurement, using the above method, whether or not CD137 positive cells could be depleted by the anti-CD137 antibody-saporin complex in in-vitro mixed lymphocyte reaction (MLR), 30 to 40% of CD137 positive T cells were observed in the T cells. After detecting CD137 expression, the respective antibodies were treated in cell culture fluid to determine the proliferation rate of the cells depending on the number of cell divisions. As a result, in the control groups, two cell divisions were observed in the CD 4⁺ cells and five cell divisions were observed in the CD8⁺ cells, whereas, the experimental groups treated with the anti-CD137 antibody and the anti-rat IgG-saporin, cell division was suppressed to a significant level in the CD4⁺ cells (2 times->0) and CD8⁺ cells (5 times->3 times). Therefore, from these results, the intracellular delivery of saporin via the anti-CD137 antibody in accordance with the present invention was highly active in selectively depleting alloantigen-specific CD137 positive T cells and suppressing cell proliferation (see FIG. 11). Also, the level of dilution of CFSE indicates the number of cell divisions. The more the cells are divided, the less the level of fluorescence of CFSE.

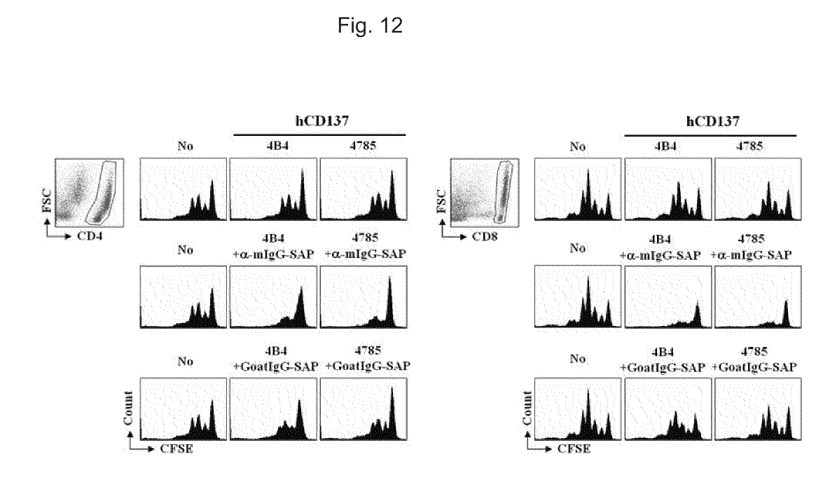

Moreover, in order to determine whether or not the CD137 positive T cells can be depleted by the anti-CD137 antibody-saporin by a human in vitron MLR method, antigen presenting cells isolated from donor peripheral blood were irradiated (3000 rads), mixed with CFSE-labeled T cells of another donor at a ratio of 1:2, and cultured in an MLR condition together with an anti-human CD3 antibody. After 24 hours, the cultured cells were harvested and the level of CD137 expression was determined. As a result, 25 to 35% of CD137 positive cells were observed in the T cells. After CD137 expression was detected, the respective antibodies were treated at a concentration of 1 μg/ml in cell culture fluid and the proliferation rate of the cells depending on the number of cell divisions was determined. As a result, as shown in FIG. 12, it was demonstrated that, in the group treated with the anti-CD137 antibody and the anti-mouse IgG-saporin, cell division was suppressed to a significant extent. Therefore, from these results, the present inventors found that the intracellular delivery of saporin via the anti-CD137 antibody of the present invention was effective in selectively depleting human alloantigen-specific CD137 positive T cells and suppressing cell proliferation.

Although the invention has been described focusing on the preferred embodiments, those skilled in the art will appreciate that the invention may be carried out in modified forms without departing from the essential characteristics of the present invention. Therefore, the above embodiments should be construed in all aspects as illustrative and not restrictive. The scope of the invention should be determined by the appended claims and their legal equivalents, not by the above description, and all changes coming within the equivalency range of the appended claims should be construed as being embraced in the invention.

What is claimed is:

1. A method for depletion of CD137 positive cells in vitro and in vivo, comprising the step of contacting an anti-CD137 antibody-toxin complex with the CD137 positive cells expressing CD137.

2. The method of claim 1, wherein the anti-CD137 antibody is an agonist antibody or antagonist antibody against CD137 molecules.

3. The method of claim 1, wherein the toxin is a chemotherapeutic agent selected from the group consisting of cyclophosphamide, melphalan, mitomycin C, bizelesin, cisplatin, doxorubicin, etoposide, mitoxantrone, SN-38, Et-743, actinomycin D, bleomycin, TLK286, SGN-15 and fludarabin; a Type I ribosome-inactivating protein selected from the group consisting of agrostin, b-32, bouganin, camphorin, curcin, gelonin, JIP60, momordin, PAP (pokeweed antiviral protein), saporin and trichosanthin; a Type II ribosome-inactivating protein selected from the group consisting of abrin, ricin, mistletoe lectin I, modeccin, volkensin, RIP, lanceolin, stenodactylin, aralin and riproximin; diphtheria toxin or venom toxin.

4. The method of claim 1, wherein the anti-CD137 antibody-toxin complex promotes apoptosis of the CD137 positive cells or suppresses proliferation of the CD137 positive cells.

5. The method of claim 1, wherein the CD137 positive cells are associated with a disease selected from the group consisting of autoimmune diseases, graft versus host diseases, transplantation, cancer, and inflammatory diseases.

6. The method of claim 1, wherein the CD137 positive cells are activated cells expressing CD137, and are selected from the group consisting of T cells, B-cells, dendritic cells, natural killer (NK) cells, macrophages, cancer cells, and myeloid cells containing neutrophils, basophils, and eosinophils.

7. The method of claim 1, wherein the anti-CD137 antibody-toxin complex is internalized into the cells by endocytosis when contacted with the CD137 positive cells.

8. The method of claim 1, wherein the toxin binds to the anti-CD137 antibody (primary antibody) or to a secondary antibody to the anti-CD137 antibody.

9. The method of claim 1, wherein the CD137 positive cells are treated with the anti-CD137 antibody-toxin complex at a concentration of 0.1 to 5.0 μg/ml.
